(19) United States
(12) Patent Application Publication
(10) Pub. No.: US 2011/0319875 A1
(43) Pub. Date: Dec. 29, 2011

(54) Apparatus and Method for Morphing a Three-Dimensional Target Surface into a Two-Dimensional Image for Use in Guiding a Laser Beam in Ocular Surgery

(76) Inventors: Frieder Loesel, Mannheim (DE); Gwillem Mosedale, Munchen (DE)

(21) Appl. No.: 13/212,012

(22) Filed: Aug. 17, 2011

Related U.S. Application Data
(63) Continuation-in-part of application No. 11/625,213, filed on Jan. 19, 2007.

(51) Int. Cl. A61F 9/008; G06T 15/20; A61B 3/14; G01S 15/89; G01B 11/30; G01B 9/02

(52) U.S. Cl. 606/4; 356/612; 356/479; 351/206; 367/88; 345/427

(57) ABSTRACT

An apparatus and method for ocular surgery includes a delivery system for generating and guiding a surgical laser beam to a focal point on a target surface in a treatment area of an eye. A detector is coupled to the beam path of the surgical laser to create a three-dimensional image of the target surface, and a computer morphs this three-dimensional image into a two-dimensional image. Operationally, the computer then uses the two-dimensional image to position and move the focal point in the treatment area for surgery.

This application is a continuation-in-part of application Ser. No. 11/625,213, filed Jan. 19, 2007, which is currently pending. The contents of application Ser. No. 11/625,213 are incorporated herein by reference.

### FIELD OF THE INVENTION

The present invention pertains generally to systems and methods for performing ocular surgery. More particularly, the present invention pertains to computer-controlled laser surgical systems. The present invention is particularly, but not exclusively, useful as a system and a method that incorporate imaging techniques for the purpose of morphing a three-dimensional treatment area into a two-dimensional image for use in controlling laser beam focal point movements within the treatment area during a surgical operation.

### BACKGROUND OF THE INVENTION

When using a laser beam to perform ocular surgery, the precise movement of the laser beam's focal point through the tissue to be altered is absolutely imperative. Specifically, focal point position accuracies within about ten microns (10 μm) are preferable. To do this, the desired path for the laser beam's focal point must have a precisely defined start point. And, the laser beam's focal point must then be moved along the prescribed path. Although this can be accomplished in some situations with open loop control (i.e. having the laser beam focal point follow a pre-programmed path), in many other situations it may be more desirable to incorporate a closed loop feedback control system. Unlike open loop systems, closed loop feedback control systems provide continuous monitoring and corrections for deviations of the focal point. In either case, movements of the laser beam's focal point must be accomplished in the context of a reference datum.

An important requirement for any closed loop feedback control system is the need to accurately identify an appropriate error signal. As implied above, this error signal must be measurable. Thus, a reference datum is required from which the error signal can be measured. Once the error signal is identified, control of the system's performance is made by system adjustments that will nullify, or at least minimize, the error signal. Stated differently, deviations (i.e. error signals) from desired performance parameters must be determinable and maintained below an acceptable minimum. For the specific case involving feedback control of a surgical laser's focal point during ocular surgery, a reference datum that is anatomically related to the eye undergoing surgery needs to be selected. Further, knowledge of the location of the laser beam's focal point relative to the reference datum, and thus relative to a path through the eye, is also required.

Anatomically, the eye includes various tissues that may be beneficially altered by laser surgery. These include: the cornea, the crystalline lens, and the retina. Importantly, a thorough knowledge of the geometry of these ocular elements, and of their geometrical relationship to each other, is essential for successful surgery. All of this, of course, cannot be done by merely an external examination of the eye. With this limitation in mind, one method for imaging the interior of an eye involves optical coherence tomography (OCT) techniques. Fortunately, these techniques are well known to skilled artisans (e.g. See U.S. Pat. No. 6,004,314 which issued to Wei et al. for an invention entitled “Optical Coherence Tomography Assisted Surgical Apparatus”). Specifically, for purposes of the present invention, OCT can be employed to identify an appropriate eye-based reference datum for conduct of the laser surgery. Further, OCT provides a means for visualizing a treatment area inside the eye, while laser surgery is being performed. Although OCT techniques may be preferred, it will be appreciated by the skilled artisan that other imaging techniques might be used for the purposes of the present invention. Specifically, imaging techniques such as confocal microscopy, or second harmonic generation microscopy, may be employed.

A consequence of the above is that the target surface (treatment area) for an ocular surgical procedure will often be three-dimensional. A useful image for guiding a laser during such surgery, however, is preferably two-dimensional.

In light of the above, it is an object of the present invention to provide a method and apparatus for directing a surgical laser beam onto tissue in a treatment area of an eye of a patient, wherein control of the laser beam is based on cross-sectional views of the eye obtained by employing OCT techniques. Another object of the present invention is to provide a method and apparatus for directing a surgical laser beam onto tissue in a treatment area of an eye of a patient wherein an eye-based reference datum can be selected that is most appropriate for the particular surgical operation that is to be performed. Yet another object of the present invention is to provide an imaging and control technique for guiding a laser beam during ocular surgery, wherein a three-dimensional target surface is morphed into a two-dimensional image that can be used to guide the laser beam. Still another object of the present invention is to provide a method and apparatus for directing a surgical laser beam onto tissue in a treatment area of an eye of a patient that is easy to implement, is relatively simple to manufacture, and is comparatively cost effective.

### SUMMARY OF THE INVENTION

In accordance with the present invention, an apparatus and a method are provided for performing ocular surgery. In particular, this surgery is accomplished by directing a laser beam onto tissue in a treatment area of a patient's eye; and it requires identifying a reference datum that is related to the eye. For purposes of the present invention, this reference datum can be either the anterior surface of the cornea, the posterior surface of the cornea, a surface area on the crystalline lens, or the retina. To identify the reference datum, the present invention employs an optical detector that creates images using optical coherence tomography (OCT) techniques. Specifically, the detector is used to create cross-sectional views of the eye that include images of both the reference datum and of the treatment area where the tissue that is to be altered by laser surgery is located.

Along with the optical detector, the apparatus of the present invention includes a beam delivery system. Specifically, the beam delivery system has a laser source for generating the surgical laser beam, and it has appropriate optical elements for directing the laser beam from the laser source to the treatment area. Included in these optical elements is a scanner that is able to move the laser beam in orthogonal x, y and z directions. Also, the delivery system includes a lens for focusing the laser beam to a focal point in the treatment area. As intended for the present invention, the surgical laser beam that is generated by the beam delivery system comprises a sequence of femtosecond pulses having a wavelength that is approximately one thousand nanometers (λₛ=1,000 nm). Preferably, the apparatus also includes a contact lens that can be placed against the anterior surface of the patient's eye, to stabilize the eye during surgery. Further, the contact lens can also establish an interface at the anterior surface between the eye and the apparatus that may be used as a reference datum.

A computer (i.e. a data processor) is electronically connected to both the beam delivery system and to the optical detector. With these connections, the computer is able to compare the location of desired focal points in the treatment area (based on pre-planned data for the surgery) with actual focal points. Deviations of actual focal points from desired focal points (i.e. error signals) can thus be identified. Using well known closed loop feedback control techniques, the delivery system is then adjusted to nullify or minimize the error signals. Consequently the system can be controlled to have its focal point follow a predetermined path through the treatment area. Alternatively, the system can be operated in an open-loop mode. If so operated, the focal point is moved to follow the predetermined path through the treatment area without any further adjustments. In the open-loop mode, however, it is still important to use the optical detector to establish an appropriate start point for the path of the focal point.

As indicated above, an important aspect of the present invention is its use of the optical detector to generate cross-sectional views of the treatment area. As envisioned for the present invention, such views can be sequentially made in real time. Further, they can be made from different perspectives, based on different cross-section planes through the eye. With these capabilities, the cross-sectional views can be used for control of the system, and they can also provide the operator with a three-dimensional visualization of the treatment area. With this capability, it is envisioned that manual control over movements of the focal point in the treatment area is possible for the present invention. When used, manual control may either augment the computer control mentioned above, or provide an alternative to the computer control.

In another aspect of the present invention, a three-dimensional target surface is morphed into a two-dimensional planar image (morph image). The morph image is used to guide the focal point of a laser beam along a predetermined path on the target surface. Specifically, the target surface is envisioned as being located inside a transparent material (e.g. an eye of a patient). Importantly, the two-dimensional planar image (morph image) of the target surface may include a visualization of a reference datum that is related to the target surface. In the case of an eye, the reference datum is preferably an anatomical feature of the eye.

Structurally, the apparatus for this embodiment for the present invention includes an energy source for generating an imaging beam. Also included is a beam delivery system for guiding the imaging beam over the target surface that is to be imaged. In cooperation with the energy source, and with the beam delivery system, a detector is used to receive reflections (returns) of the imaging beam from the three-dimensional target surface. A computer then uses these reflections (returns) to establish a three-dimensional dataset. This three-dimensional dataset is then used by the computer as input to create a two-dimensional image of the target surface. In this combination, the computer is connected to both the beam delivery system and to the detector. The consequence of this interaction is to morph the image of the three-dimensional target surface into a two-dimensional planar image of the target surface.

As indicated above, the apparatus of the present invention further comprises a laser unit for generating a laser beam. Operationally, the focal point of the laser beam is positioned by the computer with reference to the morph image, and the focal point is guided over the target surface relative to the reference datum that is included in the morph image.

By way of example, in accordance with the present invention, a methodology for morphing a target surface into the two-dimensional planar image (morph image) of the target surface is accomplished electronically. Specifically, this is done by the computer in accordance with a computer program. In sequential order, according to the computer program, the target surface is first subdivided into a plurality of three-dimensional sections. Because these sections collectively represent the entire target surface, they all will necessarily have a collective contiguity that is defined by the target surface. With this in mind, each section is individually projected onto a plane to create a two-dimensional section image. The resulting plurality of two-dimensional section images are then organized by the computer program to re-establish the collective contiguity of the sections. Thus, the two-dimensional planar image of the target surface is created for an intended use by the present invention, such as in an ocular surgery procedure.

As envisioned for the present invention, the imaging beam may be either an electromagnetic wave, or an ultrasound wave. In the case of ultrasound waves, an ultrasound device of a type well known in the pertinent art can be used. In the case of an electromagnetic wave, the two-dimensional planar image of the target surface is created using techniques such as optical coherence tomography (OCT), confocal imaging, Scheimpflug principle imaging or second harmonic generation imaging. Further, it is appreciated by the present invention that the target surface will most likely be uneven and non-planar. Accordingly, the present invention is adaptable to individually and/or collectively accommodate target surfaces having elliptical shapes, cylindrical shapes, spherical shapes, irregular shapes with discontinuities, or volumetric surfaces.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic drawing of an apparatus for performing ocular surgery in accordance with the present invention;

FIG. 2 is a top plan view of an eye as would be seen along the line 2-2 in FIG. 1;

FIG. 3 is a cross-section view of an eye as seen along the line 3-3 in

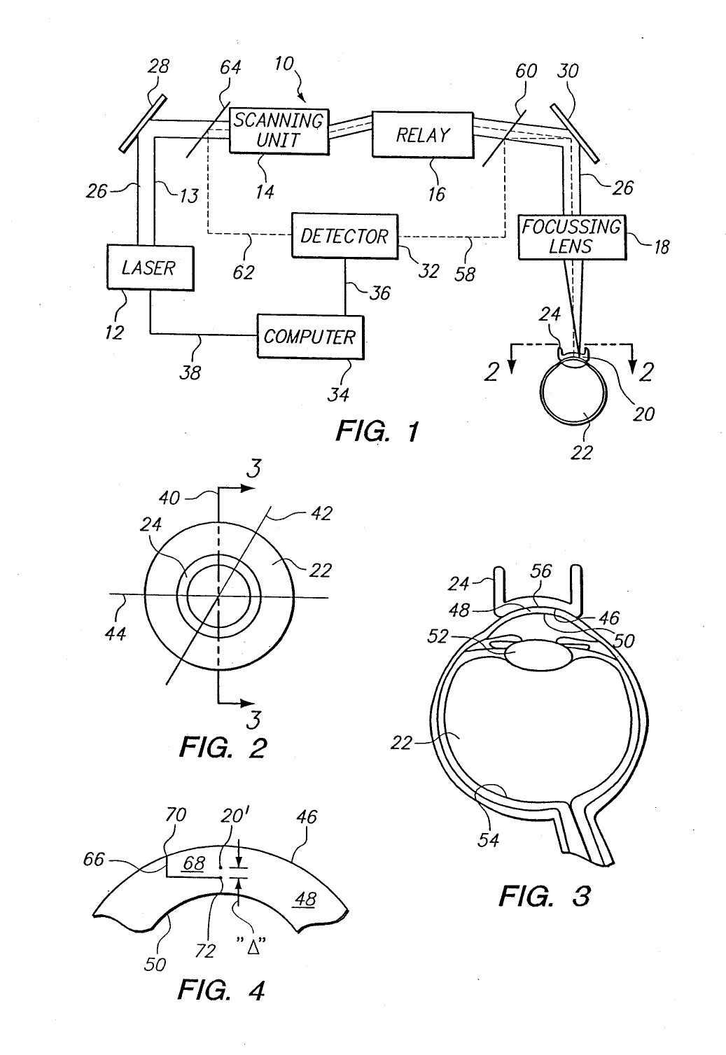

FIG. 2;

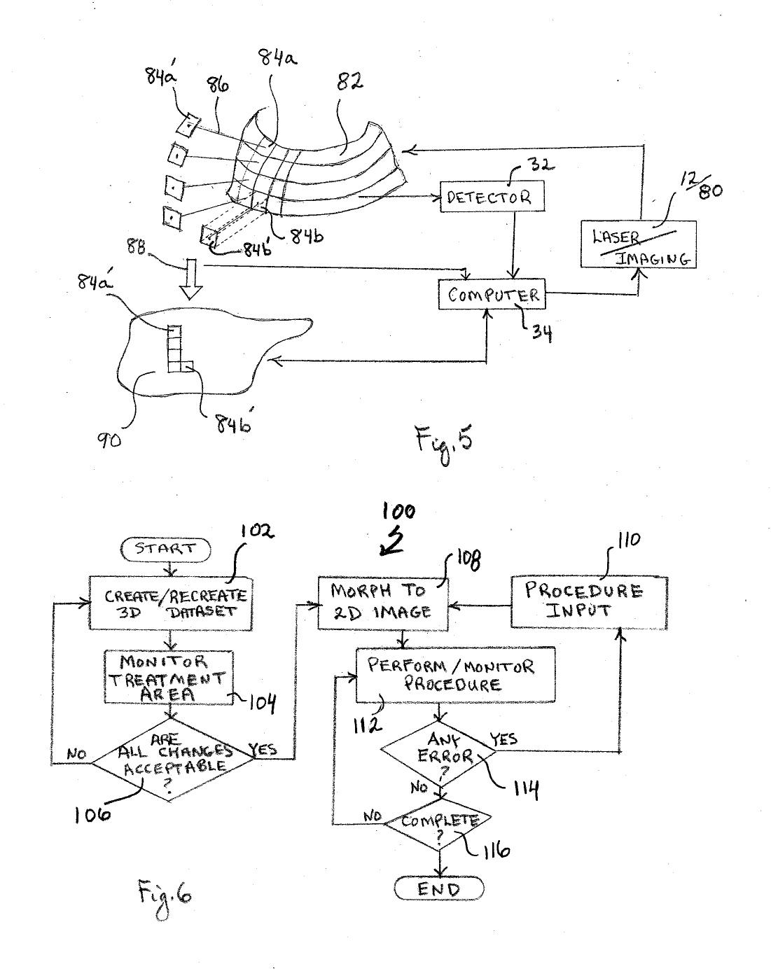

FIG. 4 is an enlarged cross-section view of the cornea of the eye shown in FIG. 3;

FIG. 5 is a schematic presentation of the functional components involved in morphing a three-dimensional target surface into a two-dimensional image; and

FIG. 6 is an operational flow chart for an interactive use of a three-dimensional dataset and a two-dimensional planar image in guiding and controlling a laser beam during ocular surgery.

### DESCRIPTION OF THE PREFERRED EMBODIMENTS

Referring initially to FIG. 1, an apparatus for performing ocular surgery in accordance with the present invention is shown and is generally designated 10. As shown, the apparatus 10 includes a laser source 12 for generating a surgical laser beam 13. For the present invention, the surgical laser beam 13 preferably includes a sequence of femtosecond pulses having a wavelength of approximately one thousand nanometers (λₛ=1,000 nm). FIG. 1 also implies that the apparatus 10 includes a scanning unit 14 that will allow the surgical laser beam 13 to be moved in orthogonal x, y and z directions. Relay optics 16 transfer the surgical laser beam 13 in a manner well known in the pertinent art, and a focusing lens 18 is used to focus the surgical laser beam 13 to a focal point 20.

As indicated in FIG. 1, the focal point 20 may be selectively established in the tissue of a patient's eye 22. A contact lens 24 that is mounted on the apparatus 10 by way of connections (not shown) is also shown positioned on the eye 22. Further, FIG. 1 indicates the surgical laser beam 13 will follow along a beam path 26 as it progresses from the laser source 12 to its focal point 20 in the eye 22. For this purpose, turning mirrors 28 and 30 can be employed to establish the beam path 26, as desired.

Still referring to FIG. 1, it will be seen that the apparatus 10 includes an optical detector 32 and a computer (data processor) 34. More specifically, the computer 34 is connected via a line 36 to the optical detector 32, and it is connected to the laser source 12 via a line 38. Together, these components (i.e. laser source 12, optical detector 32, and computer 34) effectively control the apparatus 10 during ocular surgery.

As envisioned for the present invention, and stated above, the optical detector 32 uses optical coherence tomography (OCT) techniques to create cross-section views of the eye 22. Importantly, these views include images of specific anatomical features of the eye 22. Moreover, optical detector 32 creates these views (with images) in a way that allows the images to be used by the computer 34 for control of the laser source 12. To better appreciate this function, refer to FIG. 2.

In FIG. 2, the eye 22 is seen in a top plan view; and it is shown with end-on indications of several reference planes 40, 42 and 44. The present invention envisions these planes 40, 42, and 44 will be generally parallel to the optical axis of the eye 22 and will extend through the eye 22. The planes 40, 42 and 44, however, are only exemplary, and their importance is best appreciated by cross referencing FIG. 2 with FIG. 3. Specifically, FIG. 3 is representative of a cross section view of the eye 22 as seen in the single plane 40. The fact that other cross section views of the eye 22 are possible (i.e. the perspectives of planes 42 and 44), allows OCT images to be collectively considered for a three-dimensional presentation of the interior of the eye 22. On the other hand, an individual image from any particular plane (e.g. plane 40, 42 or 44) will, by itself, provide valuable information for the use and operation of apparatus 10.

With specific reference now to FIG. 3 it will be seen that the cross section view presented (i.e. plane 40) specifically reveals several anatomical features of the eye 22. These include: the anterior surface 46 of the cornea 48, the posterior surface 50 of the cornea 48, the crystalline lens 52, and the retina 54. Further, this cross section view also shows details of the contact lens 24, if used. Thus, the interface between contact lens 24 and the anterior surface 46 of cornea 48 can be identified. At this point it is to be noted that less than an entire cross section view (e.g. as shown in FIG. 3) can be used for the purposes of the present invention. For example, an image emphasizing the cornea 48 or the retina 54 may be needed. Further, it is also to be noted that, particular information from an image (e.g. plane 40) can be substantiated or verified by comparing it with images from other planes (e.g. planes 42 or 44).

For purposes of disclosure, the interface between contact lens 24 and the anterior surface 46 of the cornea 48 is hereafter referred to as a reference datum 56. It must be appreciated, however, that this reference datum 56 is only exemplary. Other anatomical features of the eye 22 can be alternatively used for the same purposes, and perhaps more effectively, depending on the requirements of the particular ocular surgery being performed.

Returning for the moment to FIG. 1, it will be seen there are two functional embodiments of the apparatus 10 that are envisioned for the present invention. The primary difference between the two embodiments is determined by the location where optical detector 32 is coupled onto the beam path 26. For both embodiments this coupling is accomplished where the diagnostic beam, used by the optical detector 32 for OCT imaging, joins the beam path 26 of the surgical laser beam 13.

For a preferred embodiment of the present invention, the diagnostic laser beam (represented by the dashed line 58 in FIG. 1) is coupled onto beam path 26 by a dichroic mirror 60. As shown, the dichroic mirror 60 is positioned downstream from the scanning unit 14. In this case, the diagnostic laser beam 58 does not pass through the scanning unit 14. Accordingly, for this preferred embodiment, the optical detector 32 needs to include its own scanning unit (not shown).

For an alternate embodiment of the present invention, the diagnostic laser beam (represented by the dotted line 62 in FIG. 1) is coupled onto beam path 26 by a dichroic mirror 64 that is located upstream from the scanning unit 14. In this case, the optical detector 32 can use the same scanning unit 14 that is being used for the surgical laser beam 13. As an operational consideration, the diagnostic laser beam 58, 62 for both embodiments will have a wavelength of approximately one thousand three hundred nanometers (λ_{d}=1,300 nm). The implication here is the embodiment wherein the diagnostic laser beam 58 is coupled downstream from the scanning unit 14, may be preferable. This is so in order to avoid the additional refinements that are required for scanning unit 14 and the relay optics 16 when two different wavelengths use the same optical elements.

### Operation

In the operation of the apparatus 10 of the present invention, a predetermined path 66 for the focal point 20 of surgical laser beam 13 is established for ocular surgery in a treatment area 68 of the eye 22 (see FIG. 4). An image (e.g. FIG. 3), or a partial image thereof, is made using the optical detector 32. Importantly, the image (partial image) needs to include both the reference datum 56 (only exemplary) and a visualization of the treatment area 68. The focal point 20 of the surgical laser beam 13 can then be directed toward a start point 70 that is selected in the context of the reference datum 56 (cross reference FIG. 3 with FIG. 4).

Open loop control of the focal point 20, as it is moved through the treatment area 68, can be achieved by merely moving the focal point 20 along the predetermined path 66 in accordance with pre-programmed instructions in the computer 34. Whenever an open-loop mode of operation is used, however, it is important that the start point 70 be accurately established, and the path 66 be precisely pre-programmed. This will require that a desired focal point 72 coincide with the start point 70, and that the path 66 be properly oriented in the treatment area 68. As envisioned for the present invention, the coincidence of the desired focal point 72 with the required start point 70 can be accomplished using information from the optical detector 32. Thus, using the start point 70, and a predetermined definition of the path 66, the apparatus 10 can be operated in an open-loop mode to perform the desired ocular surgery. On the other hand, closed loop control may be more appropriate for the particular ocular surgery being performed. In this case, the optical detector 32 is activated to provide continuous updates of cross-section images from the eye 22. As indicated in FIG. 4, information contained in such cross-section images will include position data, relative to the reference datum 56, of both an actual focal point 20′, and a desired focal point 72 on the path 66. The positional difference “Δ” between the points 20′ and 72 will then represent an error signal that can be used for appropriate adjustments of the apparatus 10. According to well known procedures and techniques (i.e. closed loop feedback control techniques), adjustments to the apparatus 10 can be input from the computer 34 that will either nullify or minimize “A” to maintain the focal point 20 on path 66 for a successful completion of the ocular surgery.

Referring now to FIG. 5, it is to be appreciated that the apparatus 10 can include an imaging unit 80 that is useable to create an image of a target surface 82 inside a treatment area 68 (shown in FIG. 4). More specifically, the target surface 82 can be diagnostically defined for a particular ocular surgical procedure, and it can be associated with a reference datum 56 as disclosed above. In this context, the apparatus 10 is intended to be useable with a vast variety of three-dimensional surface shapes, to include elliptical shapes, cylindrical shapes, spherical shapes, irregular shapes with discontinuities, and combinations of these various shapes that may define a variety of volumetric surfaces. Importantly, the target surface 82 will likely be uneven, and is therefore three-dimensional. In the event, it is also important that the imaging unit 80 be able to properly image the target surface 82. For this purpose, the present invention envisions that the imaging unit 80 may selectively use imaging techniques such as optical coherence tomography (OCT), confocal imaging, Scheimpflug principle imaging or second harmonic generation imaging. Further, as indicated in FIG. 5, the laser source 12 may be alternately used as the imaging unit 80. Additionally, it is anticipated that ultrasound techniques may be used for the imaging purposes of the present invention.

As shown in FIG. 5, the computer 34 is connected directly to both the laser/imaging unit 12/80, and to the detector 32. The purpose for this interconnection is to establish a three-dimensional (3D) dataset that defines the entire target surface 82. The computer 34 then manipulates this 3D dataset to establish a 2D base for the purpose of guiding the focal point 20 of laser source 12 during ocular surgery. All of this is accomplished by several interrelated tasks that are accomplished in accordance with a computer program.

Based on the three-dimensional dataset that is obtained for the target surface 82, the computer program of the computer 34 subdivides the target surface 82 into a plurality of three-dimensional sections 84, of which the sections 84a and 84b shown in FIG. 5 are exemplary. It will be appreciated that all of the sections 84 have a collective contiguity. Thus, they are connected to at least one other section 84, and they all, collectively, have a common boundary that surrounds the target surface 82. At this point, the data for each of the sections 84 still define a three-dimensional surface.

Once the sections 84 of target surface 82 have been subdivided by the computer program of computer 34, they are projected onto a respective plurality of two-dimensional sections 84′. Thus, for example, the two-dimensional section 84a′ corresponds to the three-dimensional section 84a, and the two-dimensional section 84b′ corresponds to the three-dimensional section 84b. For purposes of the present invention, such projections can be done in any of several ways; all known in the pertinent art. The projection line 86 shown connecting the center of three-dimensional section 84a with the center of two-dimensional section 84a′, is only exemplary. As more specifically indicated for the projection of the three-dimensional section 84b onto the two-dimensional section 84b′, the projection of each section 84 is accomplished point-by-point.

Arrow 88 is shown in FIG. 5 to indicate that the computer 34 is used to organize the plurality of two-dimensional sections 84′ into a morph image 90. Importantly, the morph image 90 results from assembling the various two-dimensional sections 84′ in a manner that reconstitutes the original collective contiguity of the three-dimensional sections 84 on target surface 82. As indicated above, all of this is done in accordance with a computer program that is run by the computer 34. Thus, in conclusion, a two-dimensional morph image 90 of the target surface 82 is created that can be used by an operator for moving the focal point 20 of the surgical laser beam 13 during ocular surgery. As will be appreciated by the skilled artisan, the target surface 82 may also be the surgical target (i.e. treatment area) for this surgery.

In FIG. 6, an operational flow chart for the present invention is shown, and is generally designated 100. More particularly, the flow chart 100 presents a sequence of functional steps that can be taken by the computer 34 for guidance and control of the surgical laser beam 13. For the present invention, this is done using a three-dimensional (3D) dataset, and the corresponding two-dimensional (2D) morph images 90 of the treatment area 68 (target surface 82). It is to be appreciated that this guidance and control can be selectively accomplished either manually or electronically, using well known “closed-loop” control techniques.

In general, flow chart 100 indicates how the conversion (i.e. morphing) of a three dimensional dataset into a two dimensional morph image 90 can be used for “closed loop” control during an ocular surgical procedure. With reference to FIG. 6, it is to be appreciated that the three-dimensional (3D) dataset is initially generated by well-known imaging techniques. More specifically, the 3D dataset is created to physically describe a treatment area 68 (i.e. target surface 82). As disclosed above, this can be done in any of several ways known in the pertinent art (see action block 102 in chart 100). The 3D dataset is then continuously monitored during the surgical procedure (see action block 104) to detect physical changes that may affect the surgical procedure in the treatment area 68 (target surface 82). For instance, an unwanted or unexpected shift or movement of anatomical tissue may occur that needs to be accounted for. Otherwise, the procedure might be compromised. In the event, any such change is checked (inquiry block 106) to determine whether it is acceptable and can be essentially ignored. On the other hand, if the change is not acceptable, inquiry block 106 indicates that the 3D dataset needs to be appropriately recreated (see action block 102).

Once an acceptable 3D dataset has been obtained for the treatment area 68 (target surface 82), it is manipulated by the computer 34 and is reconstructed as a morph image 90 (see action block 108). Additionally, action block 110 indicates that the procedural requirements for an ocular surgical procedure are also provided as input for incorporation and presentation with the morph image 90. In essence, this incorporation establishes how the laser beam 13 will be guided and controlled during the procedure. For example, the particular path that is to be followed by the focal point 20 of the laser beam 13 can be electronically presented with the morph image 90.

Action block 112 indicates that a selected ocular surgical procedure is performed by computer 34. For the present invention this requires use of a computer program that includes information which is gleaned from both the 3D dataset and its resultant morph image 90. The closed loop capabilities for an operation of the present invention are represented by the inquiry block 114. Specifically, inquiry block 114 indicates that any detected errors in the performance of a procedure are handled and corrected by referring back to the procedural input (see block 110). Inquiry block 116 then monitors the procedure to determine when it has been completed.

While the particular Apparatus and Method for Morphing a Three-Dimensional Target Surface into a Two-Dimensional Image for Use in Guiding a Laser Beam in Ocular Surgery as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. An apparatus for creating an image of an uneven surface inside a transparent material, the apparatus comprising:
a source for generating an imaging beam;
a beam delivery system for guiding the imaging beam over the uneven surface to be imaged;
a detector for receiving a reflection (return) of the imaging beam from the uneven surface to create an image thereof; and
a computer connected to the beam delivery system and to the detector for morphing the image of the uneven surface into a two-dimensional planar image of the uneven surface.

2. An apparatus as recited in claim 1 wherein the computer includes a computer program for morphing the image of the uneven surface, and wherein the computer program comprises:
a means for subdividing the uneven surface into a plurality of sections having a collective contiguity;
a means for projecting each section in the plurality of sections onto a plane to create a respective two-dimensional section image; and
a means for organizing the resulting plurality of section images to re-establish the collective contiguity of the sections to present the two-dimensional planar image of the uneven surface.

3. An apparatus as recited in claim 1 wherein the two-dimensional planar image of the uneven surface includes a visualization of a reference datum related to the uneven surface, and the apparatus further comprises a laser unit for generating a laser beam, wherein the computer uses the image of the uneven surface to position the focal point of the laser beam on the uneven surface relative to the reference datum and to guide the laser beam over the uneven surface.

4. An apparatus as recited in claim 3 wherein the transparent material is an eye of a patient, wherein the reference datum is related to an anatomical feature of the eye, and wherein the laser beam is guided during an ocular surgery procedure.

5. An apparatus as recited in claim 1 wherein the imaging beam is an electromagnetic wave.

6. An apparatus as recited in claim 1 wherein the imaging beam is an ultrasound wave.

7. An apparatus as recited in claim 1 wherein the uneven surface is appropriately selected, individually and collectively, from a group comprising elliptical shapes, cylindrical shapes, spherical shapes, irregular shapes with discontinuities, and volumetric surfaces.

8. An apparatus as recited in claim 1 wherein the two-dimensional planar image of the uneven surface is created using techniques selected from a group comprising optical coherence tomography (OCT), confocal imaging, Scheimpflug principle imaging and second harmonic generation imaging.

9. An apparatus for creating a morph image of a target surface inside an eye of a patient which comprises:
a source for generating an imaging beam to collect a three-dimensional dataset, wherein the dataset includes information pertinent to a visualization of the target surface; and
a computer for receiving the dataset, and morphing the dataset in accordance with a computer program to create a morph image of the target surface, wherein the morph image and the target surface are heterotypic.

10. An apparatus as recited in claim 9 wherein the computer program comprises:
a means for subdividing the target surface into a plurality of sections having a collective contiguity;
a means for projecting each section in the plurality of sections onto a plane to create a respective two-dimensional section image; and
a means for organizing the resulting plurality of section images to re-establish the collective contiguity of the sections to present the morph image of the target surface.

11. An apparatus as recited in claim 10 wherein the morph image is a two-dimensional planar image.

12. An apparatus as recited in claim 11 wherein the morph image of the target surface includes a visualization of a reference datum related to an anatomical feature of the eye, and the apparatus further comprises a laser unit for generating a laser beam, wherein the computer uses the morph image of the target surface to position the focal point of the laser beam on the target surface relative to the reference datum and to guide the laser beam over the target surface.

13. An apparatus as recited in claim 9 wherein the imaging beam is an electromagnetic wave.

14. An apparatus as recited in claim 9 wherein the imaging beam is an ultrasound wave.

15. An apparatus as recited in claim 9 wherein the target surface is appropriately selected, individually and collectively, from a group comprising elliptical shapes, cylindrical shapes, spherical shapes, irregular shapes with discontinuities, and volumetric surfaces, and wherein the morph image of the target surface is created using a technique selected from a group comprising optical coherence tomography (OCT), confocal imaging, Scheimpflug principle imaging and second harmonic generation imaging.

16. A method for creating an image of an uneven surface inside a transparent material, the method comprising the steps of:
generating an imaging beam;
guiding the imaging beam over the uneven surface to be imaged;
receiving information in the imaging beam from the uneven surface, wherein the information is a three-dimensional dataset defining the uneven surface; and
morphing the three-dimensional dataset for the image of the uneven surface into a two-dimensional planar image of the surface.

17. A method as recited in claim 16 wherein the transparent material is an eye of a patient and the method further comprises the steps of:
providing a laser unit for generating a laser beam;
directing the laser beam to a focal point on the uneven surface;
including a visualization of a reference datum in the three-dimensional dataset, wherein the reference datum is related to an anatomical feature of the eye; and
guiding the focal point relative to the reference datum over the uneven surface during an ocular surgery procedure.

18. A method as recited in claim 16 further comprising the step of presenting a planned movement of the focal point on the two-dimensional planar image before the guiding step.

19. A method as recited in claim 16 wherein the morphing step comprises the steps of:
subdividing the target surface into a plurality of sections having a collective contiguity;
projecting each section in the plurality of sections onto a plane to create a respective two-dimensional section image; and
organizing the resulting plurality of section images to re-establish the collective contiguity of the sections to present the morph image of the target surface.

20. A method as recited in claim 19 further comprising the steps of:
appropriately selecting, individually and collectively, a shape for the target surface from a group comprising elliptical shapes, cylindrical shapes, spherical shapes, irregular shapes with discontinuities, and volumetric surfaces; and
creating the morph image of the target surface using a technique selected from a group comprising optical coherence tomography (OCT), confocal imaging, Scheimpflug principle imaging and second harmonic generation imaging.

21. A computer system for guiding and controlling the movement of a laser beam focal point along a path in the eye of a patient which comprises:
a means for imagining a three-dimensional surface of tissue in an eye to detect and account for physical changes in the three-dimensional surface;
a means for morphing an image of a portion of the three-dimensional surface into a two-dimensional morph image of a target surface;
a means for incorporating procedural information into the two-dimensional morph image for use in guiding and controlling the movement of a focal point of a laser beam along a path in the target surface; and
a means for minimizing errors in the movement of the focal point.

22. A computer system as recited in claim 21 further comprising a laser source for generating the laser beam.

23. A computer system as recited in claim 22 further comprising adaptive optics connected to the laser source for establishing the focal point of the laser beam.

24. A computer system as recited in claim 21 further comprising:
an imaging device for irradiating the three-dimensional surface with energy; and
a detector for receiving reflections of the energy from the three-dimensional surface to create an image of the three-dimensional surface.

25. A computer system as recited in claim 24 wherein the energy is an electromagnetic wave.
